SEQUENCE LISTING (27 sequences)

SEQ ID NO: 1 - DNA, 38 bp, Artificial Sequence
  Pseudomonas TOL plasmid Pm/Pu promoter variant
tctagaaaggcctaccccttaggctatatgcaactagt

SEQ ID NO: 2 - DNA, 38 bp, Artificial Sequence
  Pseudomonas TOL plasmid Pm/Pu promoter variant
tctagaaaggcctaccccttaagctttatgcaacgagt

SEQ ID NO: 3 - DNA, 38 bp, Artificial Sequence
  Pseudomonas TOL plasmid Pm/Pu promoter variant
tctagaaaggcctaccccttacgctttatgcaactagt

SEQ ID NO: 4 - DNA, 38 bp, Artificial Sequence
  Pseudomonas TOL plasmid Pm/Pu promoter variant
tctagaaaggcctaccccttaggttttatgcaactagt

SEQ ID NO: 5 - DNA, 38 bp, Artificial Sequence
  Pseudomonas TOL plasmid Pm/Pu promoter variant
tctagaaaggcctaccccttataatttatgcaactagt

SEQ ID NO: 6 - DNA, 38 bp, Artificial Sequence
  Pseudomonas TOL plasmid Pm/Pu promoter variant
tctagaaaggcctaccccttaagctatatgcaactagt

SEQ ID NO: 7 - DNA, 38 bp, Artificial Sequence
  Pseudomonas TOL plasmid Pm/Pu promoter variant
tctagaaaggcctaccccttacgttttatgcaactagt

SEQ ID NO: 8 - DNA, 70 bp, Artificial Sequence
  Pseudomonas TOL plasmid Pm/Pu promoter variant
tctagaaaggcctaccccttaggctttatgcaactagaaaggcctaccccttaggctttatgcaactagt

SEQ ID NO: 9 - DNA, 38 bp, Artificial Sequence
  Pseudomonas TOL plasmid Pm/Pu promoter variant
tctagaaaggcctaccccttaggctttatgcaactagt

SEQ ID NO: 10 - DNA, 16 bp, Artificial Sequence
  A primer
gcaatttaactgtgat

SEQ ID NO: 11 - DNA, 27 bp, Artificial Sequence
  A primer
tcctggtaccttttctacggggtctga

SEQ ID NO: 12 - DNA, 59 bp, Artificial Sequence
  A primer
ctagaaaggcctaccccttaggctttatgcaactagtacaataataatggagtcatgca

SEQ ID NO: 13 - DNA, 57 bp, Artificial Sequence
  A primer
tatgcatgactccattattattgtactagttgcataaagcctaaggggtaggccttt

SEQ ID NO: 14 - DNA, 32 bp, Artificial Sequence
  A primer
ctagttgcataaattataaggggtaggccttt

SEQ ID NO: 15 - DNA, 32 bp, Artificial Sequence
  A primer
ctagaaaggcctaccccttataatttatgcaa

SEQ ID NO: 16 - DNA, 20 bp, Artificial Sequence
  A primer
aagaagcggatacaggagtg

SEQ ID NO: 17 - DNA, 20 bp, Artificial Sequence
  A primer
ctcaaggatcttaccgctgt

SEQ ID NO: 18 - DNA, 20 bp, Artificial Sequence
  A primer
gggtgagcaaaaacaggaag

SEQ ID NO: 19 - DNA, 27 bp, Artificial Sequence
  A primer
ttgtactagttgcatatagcttaaggg

SEQ ID NO: 20 - DNA, 27 bp, Artificial Sequence
  A primer
ttgtactagttgcataaaacgtaaggg

SEQ ID NO: 21 - DNA, 20 bp, Artificial Sequence
  A primer
tactacgcacattggcactg

SEQ ID NO: 22 - DNA, 21 bp, Artificial Sequence
  A primer
cgtcttccatatggatccggg

SEQ ID NO: 23 - DNA, 29 bp, Artificial Sequence
  A primer
aaaggacaaacatatggcaatccacaatc

SEQ ID NO: 24 - DNA, 25 bp, Artificial Sequence
  A primer
atcagggaattctgtgtttgtcata

SEQ ID NO: 25 - DNA, 32 bp, Artificial Sequence
  A primer
ctagttgcataaagcctaaggggtaggccttt

SEQ ID NO: 26 - DNA, 32 bp, Artificial Sequence
  A primer
ctagaaaggcctacccctnnnnnnnnnnncaa

SEQ ID NO: 27 - DNA, 69 bp, Artificial Sequence
  Transcriptional/translational region of the Pm promoter
ctatctctagaaaggcctaccccttaggctttatgcaactagtacaataataatggagtcatgcatatg

(19) United States
(12) Patent Application Publication
(10) Pub. No.: US 2003/0003525 A1
(43) Pub. Date: Jan. 2, 2003

(54) Novel promoter of gene expression

(76) Inventors: Svein Valla, Trondheim (NO); Hanne Winther-Larsen, Oslo (NO)

Correspondence Address: SCHWEGMAN, LUNDBERG, WOESSNER & KLUTH, P.A., P.O. BOX 2938, MINNEAPOLIS, MN, 55402, US

(21) Appl. No.: 10/012,898

(22) Filed: Nov. 5, 2001

Related U.S. Application Data
(63) Continuation of application No. PCT/GB00/01729, filed on May 5, 2000.

(30) Foreign Application Priority Data
May 5, 1999 (GB) 9910413.5

(51) Int. Cl. C12Q 1/18; C07H 21/04; C12N 1/21; C12N 15/74

(52) U.S. Cl. 435/32; 435/252.3; 435/320.1; 536/23.2

(57) ABSTRACT

The invention provides an isolated DNA molecule comprising a promoter sequence, said promoter sequence being a Pm or a Pu promoter of a TOL plasmid, said promoter sequence having sequence modifications in its 10 region together with DNA constructs, expression vectors and transformed cells containing such molecules. Also provided by the invention is a method for assaying promoter activity, comprising expressing in an antibiotic-susceptible (i.e. sensitive) host cell, an antibiotic resistance gene under the control of the promoter to be assayed, and assessing the growth of said cell in the presence of said antibiotic.

The present invention relates to novel mutants of the TOL plasmid Pm or Pu promoter which facilitate gene-independent expression enhancement, reduction and/or improved regulatory control of recombinant gene expression in a broad range of expression vectors and host cell types. In particular, the Pm and Pu promoter mutants of the present invention differ from the native Pm/Pu sequence in the region of the DNA-dependent RNA polymerase binding site which lies upstream of the transcriptional start site, ie. in the so-called −10 region.

The cloning and expression of genes is a central tool in biotechnology. Traditionally, genes have been cloned and expressed in enteric bacteria, most notably E. coli, which for a long time was regarded as the most useful host for gene cloning. However, the inability of E. coli to express some biological properties, for example certain metabolic activities, or to carry out appropriate modifications and processing of gene products, e.g. by post-translational modification, has created a need for the development of alternative host-vector systems.

The ability to select expression vectors which are capable of controlled expression in non-enteric cell types, realises the possibility of tailoring cellular metabolic activities to specific end products, for example, heterologous gene expression, metabolic pathway control, protein engineering, to mention but a few. The use of non-enteric bacteria for basic and applied molecular research has extended the need for well characterised vector systems for use in such organisms. Thus, vector systems have been designed which are specific for the bacterial species of interest, e.g. soil bacteria. However, a more useful approach would be to design vectors which may be used across a broad range of microbial hosts, and work in recent years has been directed to this end.

In addition, expression of foreign genes, and indeed manipulation of the expression of native genes, can significantly perturb the physiology of the host cell and constitute a strong selective pressure for elimination or inactivation of the cloned genes. The expression control elements incorporated into expression vectors which facilitate the regulation of heterologous expression of cloned genes, may be of importance in maximising the efficiency and control of gene expression and thus of biotechnological processes.

Thus, to maximise the potential for biotechnology using recombinant DNA expression, different types of regulatory control elements are desirable. For example, where a promoter system is used to control the expression of a product which is to be harvested from the cells or culture medium, a high yield of product is desirable and hence a high level of gene expression is required.

If the regulatory elements are controlling the expression of a gene, the product of which controls or is involved in a single step of a metabolic pathway, requiring only a moderate or perhaps very low level of expression, then clearly high levels of induced transcription are not desirable, and low levels of expression are to be aimed for.

In the control of metabolic pathways by biotechnological means, there may often be a requirement for different levels of recombinant DNA expression occurring simultaneously within a cell, such expressed genes and regulatory elements being on the same or different vectors which may be extrachromosomal or integrated into the host genome for example, as in the case of transposons. Clearly it is desirable that a bank or pool of regulatory control elements is available to the molecular biologist with differing levels of potential for expression in order to fulfil the multiplicity of different functions to which such regulatory elements may be applied.

Even more important in many respects than the actual level of expression possible from a promoter system, is the level of control achievable from promoter systems, such that the inducibility and repression of the system can be tightly controlled and any non-specifically induced expression or “leakage” can be minimised or preferably eliminated altogether. Such control is important for example, for minimising the extra metabolic load imposed on cells whilst growing, allowing an efficient cell growth phase before expression of the recombinant gene. Alternatively, one or more steps in a multi-step biosynthetic pathway may be “turned on and off ” as desired by using a tightly controlled promoter system.

The present invention is directed towards meeting this continuing need for new and improved expression regulatory elements for the controlled expression of genes in a wide range of expression vectors, host organisms and cell types.

The TOL plasmids are a series of well-characterised naturally occurring plasmids and their derivatives, which occur in Pseudomonas sp. and which encode the enzymes required for the catabolism of toluene and xylenes. The TOL plasmid pWWO of Pseudomonas putida may be regarded as an archetypal TOL plasmid (for a review see Assinder and Williams 1990, Adv. Microb. Physiol., 31, 1-69).

The catabolic genes of TOL plasmids are organised in two operons, an upper pathway operon (OP1) encoding genes and regulatory sequences required for the oxidation of aromatic hydrocarbons to aromatic carboxylic acids, and a lower, or meta pathway operon (OP2) necessary for the oxidation and ring cleavage of the aromatic nucleus of aromatic carboxylic acids, giving rise to intermediates which are channelled into the intermediary metabolism. The expression of the two operons is controlled by two positive regulatory proteins XylR and XylS, in the presence of the corresponding substrate ligands toluene/xylene and benzoate/toluate respectively. Activated XylR stimulates transcription from the Promoter Pu of the upper pathway operon, whereas activated XylS induces the meta pathway operon from the promoter Pm. XylR may also induce the promoter Ps of the xylS gene (see Assinder and Williams, supra).

The binding site for activated XylS is believed to be a motif localised in the region −40 to −70 nucleotides upstream of the transcriptional start site (Gonzalez-Perez et al. (1999) J. Biol. Chem. 274(4):2286-2290). Initiation of transcription was initially believed to be mediated by the DNA dependent RNA polymerase (hereinafter referred to as RNA-pol) and the XylS/inducer complex in association with either σ⁷⁰, in the early exponential growth phase, and later by σ^{S}. However, it has recently been clarified that it is σ³² rather than σ⁷⁰ which is involved in exponential phase, and σ³⁸ in stationary phase (Marques et al., 1999, Molecular Microbiology, 31(4), 1105-1113). Overproduction of XylS also activates expression from Pm in the absence of inducer.

The TOL regulatory functions Pu/xylR or Pm/xylS have been combined with minimum replicons of the broad host range RK2-based replicon within an expression vector construct as fully disclosed in WO98/08958. This disclosure provides vectors useful for high and low level inducible expression of cloned genes using the native promoter in Pu/xylR or Pm/xylS promoter systems. The minimal RK2 replicons comprise the origin of vegetative replication oriV and the gene encoding the essential replication initiation protein TrfA that binds to iterons in oriV. Specific point mutations in the trfA gene result in an up to 24-fold increase in the copy number of the plasmid, leading to enhanced levels of gene expression from the Pm/xylS promoter system.

Mutations made to study XylS binding to Pm have previously been reported. In particular, Kessler et al. (1993) J. Mol. Biol 230: 699-703 describes mutants of the Pm system which exhibit modified and in particular ‘expression down’ characteristics by virtue of point mutations and deletions in the XylS binding site of the Pm promoter, which may overlap with the −35 region of the promoter. As indicated above, the XylS binding site of pm is known to confer activated XylS sensitivity to the Pm promoter, effectively ‘switching’ expression of the native or heterologous gene downstream of the Pm promoter and under the control of Pm, on and off. Hence, alterations in the control of inducibility or level of expression achievable from the promoter could be expected to be influenced by sequence alterations in the XylS binding region of the operon, as is borne out in Kessler et al., supra and indeed may be expected at any site in the −40 to −70 region as defined by Gonzales-Perez et al., (1999), supra.

Surprisingly though, the applicants have found that mutations downstream of the −40 to −70 region of XylS sensitivity, in a region overlapping the region of −10 nucleotides upstream of the transcription start site and to which the RNA pol binds, results in significant alterations to the level of expression achievable from the Pm promoter system relative to native Pm. Such alterations in expression may be in terms of both enhanced expression, so-called ‘expression-up’ mutants and reduced expression, so-called ‘expression-down’ mutants.

Even more surprising is the finding that some mutants in the −10 region of the Pm promoter show greatly reduced leakage or non-specifically induced ‘background’ expression.

Analogous mutations in the −10 region of the Pu promoter may similarly be made.

In a first aspect therefore, the present invention provides a promoter derived from the Pm or the Pu promoter of a TOL plasmid wherein said promoter is modified in the −10 region.

More particularly, this aspect of the invention provides a DNA molecule comprising a promoter sequence, said promoter sequence being a Pm or a Pu promoter of a TOL plasmid, and having sequence modifications in its −10 region.

Thus, the invention provides novel promoters which are mutants in the −10 region of the TOL plasmid Pm or Pu promoter. The novel promoters of the invention may be used to form novel and useful promoter systems based on the TOL plasmid regulatory elements or functions. Such a promoter system may advantageously comprise a promoter according to the invention and a regulatory gene xylS or xylR.

Thus, a further aspect of the invention provides a DNA construct comprising a modified Pm or Pu promoter as defined above together with a corresponding regulatory gene xylS or XylR.

A still further aspect of the present invention provides an expression cassette comprising a modified Pm or Pu promoter as defined above together with a corresponding regulatory gene xylS or xylR.

As used herein the term “expression cassette” refers to a nucleotide sequence encoding or comprising the various functions required to express a DNA sequence, notably the promoter-operator functions and the associated regulatory sequences required for expression from that promoter, e.g. translational and transcriptional control elements and/or sequences encoding regulatory proteins, which may act to regulate expression, for example at the level of the promoter.

As explained above, and discussed in more detail below, the TOL pasmids and their regulatory elements including Pm/xylS and Pu/xylR are well known and well-characterised in the art and in the literature.

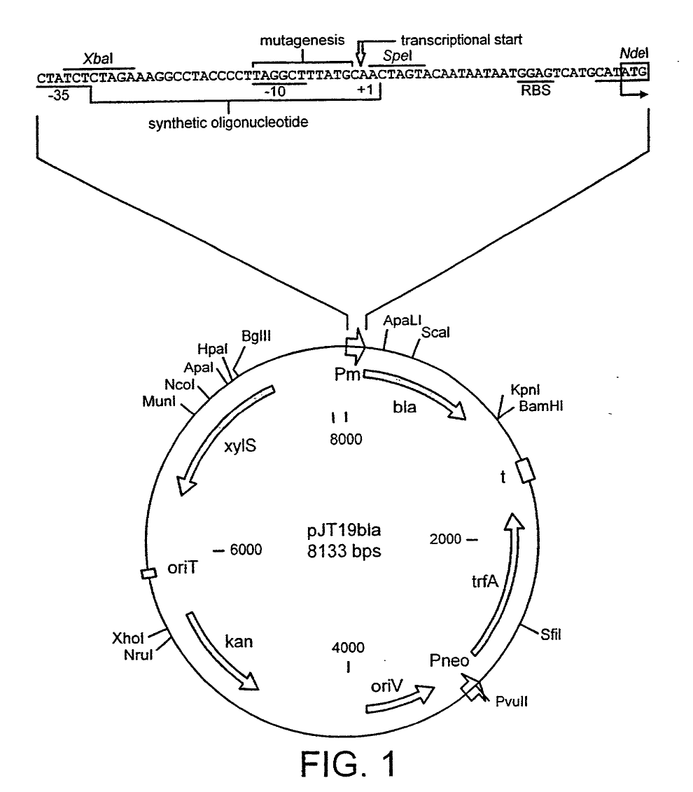

By the “−10 region”, is meant the region surrounding or flanking the 10th nucleotide situated immediately upstream of the transcription start site of a Pm or Pu promoter. With regard to the Pm promoter, reference is made to FIG. 1 which shows the transcription start site as +1 (the start site is the first nucleotide of a transcribed DNA sequence and is denoted as +1; the nucleotide preceding the start site is denoted as −1) (see also Kaldalu et al. , 1996, Mol. Microbiol. 20: 569-579 and Kessler et al., 1993, J. Mol. Biol. 230: 699-703). The transcription start site (+1) for Pu is shown in Inoye et al., Proc. Natl. Acad. Sci., USA, 81, 1688-1691.

Broadly speaking, the −10 region is to be understood to cover the region spanning the nucleotide sequence from −1 to −25 (more particularly −1 to −20 or −1 to −17) nucleotides upstream of the transcriptional start site and is the area of the double helix to which the RNA pol becomes chiefly associated upon DNA binding prior to the initiation of transcription. The −10 region is ‘recognised’ by the RNA pol on the basis of its nucleotide sequence dependent 3-dimentional conformation which is exposed to the RNA pol when the DNA helix is twisted by torsion-induced influences further upstream of the −10 region. Such torsion-induced changes and recognition of the −10 region are generally the result of the binding of the activated inducer, in this case activated XylS or XylR, upstream of the −10 region. Preferably, the −10 region spans the nucleotide sequence from −1 to −17 bases, more preferably −2 to −15 or −2 to −12. As will be described in more detail below, mutants may advantageously be created comprising modifications in the region −6 to −12, or more particularly in the region −7 to −12, or −6 to −10 from the the transcription start site.

The terms “modified” and “sequence modification” as used herein in relation to the promoters are intended to define promoters which differ in nucleotide sequence from the native (wild-type) Pm or Pu promoter sequence in the −10 region. In other words in a modified promoter, the nucleotide sequence in the −10 region as defined above is different to the nucleotide sequence of the corresponding region of a wild-type promoter. Likewise “sequence modifications” mean the sequence in the −10 region is altered or different over the wild-type. Such modifications may be generated by mutagenesis and may be random or site-directed. Random mutagenesis may be induced by chemical crosslinking agents or by radiation, for example exposure to UV light, or may involve chemical modification of the nucleotides.

Modification of the nucleotide sequence includes addition, deletion or substitution of single or multiple nucleotides, and may involve repetition (e.g. duplication) or inversion of fragments comprising two or more nucleotides within the −10 region. Included within the sequence modifications encompassed by the present invention are multiplications e.g. duplications of the entire −10 region as defined above (as well as of fragments thereof). Thus a “modification” according to the invention may include a repeat (e.g. 2 to 6, or 2 to 4 repeats) of the −10 region or a fragment thereof. Furthermore, the repeated sequence may also contain one or more base changes.

Also included are modifications which involve replacement of all or a portion of the −10 region with another sequence. For example, the entire −10 region as defined above may be replaced by a −10 region from another promoter. One such embodiment may be represented by a modified Pu promoter in which the wild type −10 region is replaced by the −10 region of a Pm promoter and vice versa.

Modifications may be introduced by whatever convenient or appropriate means (e.g. base substitution, insertion etc.) to introduce desired features into the mutant promoter of the invention, for example to introduce a consensus σ⁷⁰ or σ³² sequence.

It will be understood, therefore, that references herein to “in the −10 region” include not only bases changes in the “−10 sequence” as defined above, but also other changes associated with the −10 region, such as substitution of the −10 region, and sequence inversions and repetitions of all or a portion of the said “−10 sequence”.

In consequence of certain changes, for example replacement with a −10 region from another promoter, or the introduction of consensus sequence or another desired feature, it may be necessary to make other modifications to the sequence to “accommodate” or compensate for the primary change. For example, it may be necessary or desirable to maintain or introduce new spacing distances, e.g. between an introduced Pm −10 region in a Pu promoter and the XylR binding site and vice versa. Such optimisation involves routine trial and error and is within the normal skill of the person skilled in the art.

As will be described in more detail in the Examples below, single or multiple base changes in the −10 region of the Pm promoter yield favourable results and in a preferred embodiment of the invention, the sequence modifications in the −10 region of a Pm or Pu promoter comprise 1 to 6, or more particularly 1 to 4 or 1 to 3 base changes which may be contiguous or non-contiguous.

Thus a variety of modifications to the −10 region are possible, either singly or in combination. Functionally speaking, what is required is that the promoter retains the ability to function as a promoter of transcription, and desirably also, the ability to the regulated by a regulatory gene, although functional characteristics, such as the level of induced expression or the level of backround expression, and/or the ratio between them may be altered.

Advantageously, in the case of modified Pm promoters, the mutants of the invention retain the ability to interact with (ie. be regulated by) XylS (or a derivative of XylS). Analogously, in the case of the Pu promoter, the mutants retain the ability to interact with XylR (or a derivative thereof).

Such modification or mutagenesis may be site-directed or introduced by the use of various different techniques, e.g. PCR-based techniques, all of which are well understood by the person skilled in the art and widely reported in the literature.

A preferred method for introducing mutations in the −10 region uses doped oligonucleotide cassette mutagenesis as is known and described in the art for example in Wells et al., (1985), Gene 34:315-323 and Zheng et al. (1988), Comput. Biol. Med. 18:409-418.

In particular, the present invention provides Pm or Pu mutant promoters, modified in the −10 region of Pm or Pu, which result in expression enhancement in the induced state, so-called “expression-up mutants”. Especially, the invention provides expression-up mutants in which the enhanced expression is gene-independent.

In a further embodiment, the present invention provides promoters exhibiting reduced expression i.e. “expression-down” mutant Pm and Pu promoters.

In a further preferred embodiment the invention provides Pm or Pu mutant promoters which result in reduced leakage or background levels of expression. Promoters with reduced leakage may exhibit either expression-up characteristics, expression-down characteristics, or may show no significant change in the levels of expression attainable relative to wild type or native Pm or Pu.

Especially preferably, the mutant promoters of the present invention, be they expression-up, expression-down, reduced backround or improved control mutants, are mutants of Pm.

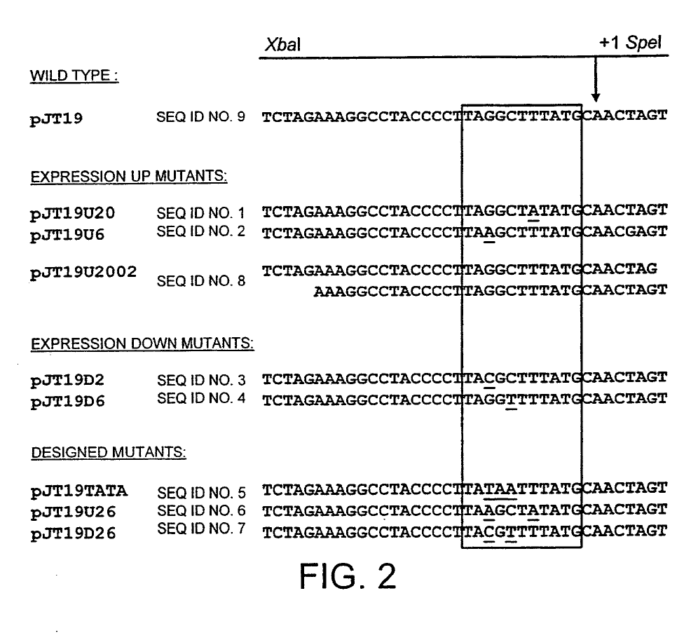

In representative embodiments, the present invention provides Pm promoter mutants comprising a sequence in the −10 region as set out, (more particularly as comprised), in any one of SEQ ID NO. 1 to SEQ ID NO. 8. This is illustrated in more detail in FIG. 2 which annotates the sequences of SEQ ID NOS. 1 to 8. The +1 start site is indicated and the 11 bp region covered by the doped oligonucleotide for mutagenesis, and comprised within the −10 region, is boxed.

The Pm/xylS system is especially advantageous due to the low cost and availability of inducer molecules and their ability to enter the host cell by passive diffusion.

In a further aspect, the present invention provides a process for preparing a mutant Pm or Pu promoter as defined above by addition, insertion, deletion or substitution of single or multiple nucleotides and/or inversion or repeat of two or more nucleotides in the −10 region thereof.

The mutant promoter systems of the invention are suitable for use in a broad range of vector types and an expression vector comprising a Pm or Pu promoter mutant which exhibits a modified nucleotide sequence as defined herein provides a further aspect of the present invention.

Such a vector may be any vector known in the art and may take the form, for example, of a plasmid, virus, transposon, phagemid or phage-derived vector, or any other replicon and may exist or function extrachromosomally in an autologously replicating form or may be integrated into the chromosome. A chromosomally-integrating vector may, for example, be in the form of a transposon, or a linearised plasmid or some other vector which recombines with host DNA thus inserting itself into the chromosome, either at a random or semi-random site or at a particular defined location by targetted integration (e.g. using homologous sequences). Shuttle vectors capable of replication and expression in both prokaryotic and eukaryotic systems, for example capable of replication and manipulation in E. coli and expression in Saccharomyes cerevisiae are also part of the invention.

A preferred embodiment of such a vector comprising the mutant Pm or Pu promoter system is the RK2-based minimum replicon as illustrated herein. Detailed information on the preparation of RK2 replicons using the non-mutant Pm or Pu promoter may be found in WO98/08958 and analogous vectors, substituting the mutant promoters for the native ones described, may analogously be prepared.

Briefly, RK2 is a well-characterised naturally occurring 6 Kb self-transmissible plasmid of the IncP incompatibility group well known for its ability to replicate in a wide range of gram-negative bacteria (Thomas and Helinski, 1989, in Promiscous Plasmids in Gram-negative bacteria (Thomas, C. M., Ed.) Chapter 1, pp 1-25, Academic Press Inc (London) Ltd, London). It has been determined that the minimal replicating unit of RK2 consists of two genetic elements, the origin of vegetative replication (oriV), and a gene (trfA) encoding an essential initiator protein (TrfA; that binds to short repeated sequences (iterons) in oriV (Schmidhauser and Helinski, 1985, J. Bacteriol. 164, 446-455; Perri et al., 1991, J. Biol. Chem; 266, 12536-12543). This minimal replicating unit is termed the so-called “RK2 minimum replicon”, and has been extensively characterised and studied in the literature. A wide range of replicons (termed “mini-RK2 replicons”) and cloning vectors based on the RK2 minimum replicon or on derivatives of the RK2 plasmid have been prepared and described in the literature (see, for example, Li et al., 1995, J. Bacteriol. 177, 6866-6873; Morris et al., J. Bacteriol., 177, 6825-6831; Franklin and Spooner, in Promiscous Plasmids in Gram-negative bacteria (Thomas, C. M., ed) Ch. 10, pp 247-267, Academic Press Inc. (London) Ltd., London; Haugan et al., 1992, J. Bacteriol 174:7026-7032; and Valla et al., 1991, Plasmid, 25, 131-136, WO98/08958).

The Pm or Pu mutant promoter may be derived from any non-mutant Pm or Pu promoter and sources for these are widely known as described in the literature. Thus a native/wild type Pm/Pu promoter may be cloned directly from its natural host, e.g. a Pseudomonas species e.g. P. aeroginosa or, for example, a Pm/Pu promoter may be derived from a synthetic source e.g. a plasmid or other vector containing a Pm/Pu promoter, for example in the case of Pm, plasmid pERD21, (a RSF1010-based replicon, Ramos et al., 1988, Febs Letters, 226, 241-246) or in the case of Pu, plasmid pRD579 (a R1-based replicon, Dixon et al., 1986, Molec. Gen. Genet. 203, 129-136) and then subjected to mutation/modification as described above. Likewise a xylS or xylR gene may similarly be obtained.

Thus, any of the TOL plasmids and their derivatives widely known and described in the literature could be used as the source of the TOL regulatory functions (see e.g. Assinder and Williams, Keil and Keil, Supra and Mermod et al., 1986, J. Bacteriol., 167, 447-454). Indeed, a number of plasmids are known in the literature which have TOL genes inserted, and any of these could be used as the source of the TOL regulatory functions for the present invention. The regulatory gene xylS/xylR may be inserted together with the Pm/Pu promoter from the same source (for subsequent mutation of Pm/Pu) or the promoter and regulatory gene may be derived independently from separate sources.

For example, to prepare an expression cassette, a xylS or xylR gene may be derived from any available source, for example in the case of xylS, plasmid pERD839 (a plasmid based on the RSF1010 replicon, Michan et al., 1992, 267, 22897-22901; this publication also mentions other plasmids which may be the source of xylS genes, e.g. pERD103 for wild-type xylS) and inserted into any appropriate vector along with a mutant Pm promoter. The xylS gene itself may be in native or mutant form. These sources are however only exemplary, and a number of alternative source plasmids could be used, selected from among the vast number known in the literature. Analogous principles apply for Pu/xylR.

Other representative plasmids which could be used, include those described in Cuskey and Sprenkle, 1980 J. Bacteriol. 170(8), 3742-3746; Mermod et al., 1986, J. Bacteriol. 167(2), 447-454; and transposon vectors are described in de Lorenzo et al., Gene, 1993, 130, 41-46.

As used herein the terms “RK2 minimum replicon” and “TOL regulatory functions” and indeed the separate genetic elements of “oriV”, “trfA” and “xylS” and “xylR” include not only the native or wild-type functions as they appear in the original, parental or archetypal source plasmids but also any modifications of the functions, for example by nucleotide addition, deletion or substitution, or indeed chemical modification of the nucleotides, which occur naturally, e.g. by allelic variation or spontaneous mutagenesis, or which are introduced synthetically. Techniques for modification of nucleotide sequences are standard and well known in the literature and include for example mutagenesis, e.g. the use of mutagenic agents or site-directed mutagenesis. PCR may also be used to introduce mutations. Appropriate or desired mutations, may for example be selected by mutant screening of the genetic element in question.

Thus, for example, expression could be increased by expressing more XylS, as described for example by Kessler et al., 1994, J. Bacteriol., 176, 3171-3176. A number of modifications of the xylS gene have also been reported, for example the xylS mutant xylS2tr6, which exhibits an altered effector specificity, and can mediate a 3-8 fold higher level of transcription than can wild-type xylS at a wide range of temperatures (Ramos et al., supra) , and the mutant gene xylSarg41pro (=xylS839), which causes a reduction in the basal transcription level from Pm, compared to wild type xylS (Michan et al., supra). All such modifications may be used according to the present invention.

It has also been found that the xylS/xylR gene may be inserted into the vectors in either orientation.

Techniques for excising the desired nucleotide sequences containing the Pm (mutant or native) promotor, regulatory regions or any other desired function from a selected source and introducing them into an expression vector or intermediate construct are well known and standard in the art, and are described for example in Sambrook et al., 1989, Molecular cloning; a laboratory manual, 2nd Edition, Cold Spring Harbour Laboratory Press, Cold Spring Harbour, N.Y.

It may be convenient to isolate the desired sequences from a selected source and introduce them, using techniques standard in the art, into a series of intermediate constructs, which may be plasmids, introducing, adding or deleting further genetic elements for example linkers, activator sites, other consensus sequences etc. to arrive at the desired expression vectors of the invention.

Functions may also be introduced to stabilise the expression vectors, or to assist in their maintenance in a broad range of hosts. Selectable markers are also usefully included in the mutant Pm/Pu promoter-containing vectors of the invention, for example to facilitate the selection of transformants. A wide range of selectable markers are known in the art and described in the literature. Any of these may be used according to the present invention and include such as the antibiotic resistance markers for example those carried by the RK2 plasmids and their derivatives, or indeed any of the TOL plasmids or their derivatives, or any other plasmid. However, properties such as sugar utilisation, proteinase production or bacteriocin production or resistance may also be used as markers. The TOL plasmid xylE structural gene may advantageously be used as a marker. This gene encodes the product C230 which may readily be detected qualitatively or assayed. Spraying a plate of bacterial colonies with catechol rapidly distinguishes C230⁺ colonies since they turn yellow due to the accumulation of 2-hydroxy muconic semialdehyde, enabling transformants/transconjugants etc. rapidly to be identified, by the presence of xylE in the vectors.

Other features which may be included in the vectors include further regulatory and/or enhancer functions, for example further transcriptional controls or translational control sequences such as start or stop codons, transcriptional initiators or terminators, ribosomal binding sites etc. Control elements such as start codons or ribosomal binding sites etc. naturally associated with the native Pm promoter may be used, or alternative or additional elements may be introduced.

In a preferred embodiment of the invention a transcriptional terminator is inserted upstream of the modified Pm/Pu promoter. As mentioned above, modifications may also be introduced into other genetic elements comprised in the vectors of the present invention. For example in RK2 type replicons, modifications may be introduced into the trfA gene, to increase copy number of the vector within a host cell. Other modifications or control elements may be introduced for example to achieve temperature sensitive replication. Such modifications have been described in the literature. The copy number of RK2 for example within E. coli is usually estimated to be 5-7 plasmids per chromosome. However, this may be elevated in both E. coli and other bacteria by certain point mutation in the trfA gene, which may lead to copy numbers up to 23-fold higher than normal which may advantageously be combined with the mutant Pm/Pu promoters in expression vectors. Such “copy up” or “cop mutations” are described for example in Durland et al., 1990, J. Bacteriol, 172, 3859-3867 and Haugan et al., 1995, plasmid, 33, 27-39.

It is of course to be understood that different modifications, additional or optional features will differ between vectors depending on their individual properties, intended function and the end use of the vector, for example incorporation of trfA into the control of the modified Pm into RK2 based vectors, as fully described in WO98/08958 for native Pm, to allow vector replication to be controlled.

As stated previously, the combined features of the RK2-based replicon and the Pu/xylR and Pm/xylS regulatory function wherein Pu and Pm exist in their native forms, are described in WO98/08958, and these known vectors serve as a useful comparison by which to assess the advantageous and improved characteristics of vectors comprising the mutant Pm/Pu promoter systems of the present invention. Precise methods for creating a RK2-based vector comprising native Pm or Pu are fully disclosed in WO98/08958 and the function and properties of such vectors are disclosed therein. Analogous vectors comprising the Pm/Pu promoter mutants of the present invention in place of wild type Pm or Pu represent one preferred embodiment of the invention.

In yet another aspect, the present invention provides host cells containing (e.g. transformed with) a vector comprising the novel Pm or Pu promoter mutants as defined herein. The vectors of the invention comprising the mutant Pm or Pu promoter and expressing genes under the regulatory control of such a promoter system, may be used to transform a wide range of cell types.

The host range of the vector will depend upon the nature of the vector construct selected. For RK2-based vectors, the host range is broad and includes a vast range of Gram-negative bacteria, as well as Gram-positive bacteria. Suitable Gram-negative bacteria include all enteric species, including, for example, Escherichia sp., Salmonella, Klebsiella, Proteus and Yersinia and non-enteric bacteria including Azotobacter sp., Pseudomonas sp., Xanthomonas sp., Caulobacter sp, Acinetobacter sp., Aeromonas sp., Agrobacterium sp., Alcaligenes sp., Bordatella sp., Haemophilus Influenzae, Methylophilus methylotrophus, Rhizobium sp. and Thiobacillus sp. Gram-positive bacterial hosts which may be used include Clavibacter sp.

Methods for introducing expression vectors into host cells and in particular methods of transformation of bacteria are well known in the art and widely described in the literature, including for example in Sambrook et al., (supra). Electroporation techniques are also well known and widely described.

In a still further aspect, the invention thus also provides a method of expressing a desired gene within a host cell, comprising introducing into said cell an expression vector as hereinbefore defined, containing said desired gene, and culturing said cell under conditions in which said desired gene is expressed.

Advantageously, the desired gene may encode a desired polypeptide product and hence the invention also provides a method of preparing such a desired polypeptide product by culturing a host cell containing an expression vector of the invention into which the desired gene has been introduced (under the control of the mutant Pm or Pu promoter), under conditions whereby said polypeptide is expressed, and recovering said polypeptide thus produced.

The cells may be transformed with and act as hosts to vectors in the form of autologously replicating extrachromosomal entities or vectors which become integrated into the chromosome, in the form of transposons, modified viruses, phage etc.

Certain properties of host cells may be particularly advantageous for expression of genes under the regulatory control of the Pm/Pu promoter of the invention. For example, if the expression cassette is integrated into the genome, the use of rec mutants to reduce the possibility of cassette excision may be advantageous, or for performing certain biotechnological ends, altered specificity of cells for substrate uptake or metabolism, or natural variations in cellular acylation, pyruvylation activity, sulphonation etc.

In a further aspect, the present invention provides a culture of cells as defined above containing vector comprising the mutant Pm or Pu promoter system.

Transcription from the mutant Pm of Pu promoter can be activated by different inducers, and different inducer compounds can lead to different levels of promoter activation (Ramos et al., 1990, J. Mol. Biol. 211, 373-382). This property may also be used to fine-tune expression levels.

It may also be possible further to modify expression levels by modifying culture conditions. Thus, the expression system may be altered by changing the growth conditions of the host cell, e.g. temperature, culture medium composition and other culture conditions such as speed of agitation, vessel size etc. Such culture modifications are known in the art. It has been found, for example, that expression from Pm increases at lower temperature.

The genes which may be expressed in the vectors under the control of the mutant Pm or Pu promoters of the invention include any desired or cloned genes including partial gene sequences, or any nucleotide sequence encoding a desired expression product, including fusion protein products, such as, for example, a desired gene sequence linked to a further nucleotide sequence encoding a further polypeptide such as β-galactosidase or glutathione-S-transferase. Such “fusion proteins” are well known in the art. The genes which are expressed under the control of mutant Pm promoter system may thus include genes which are heterologous or homologous to the host cell. The genes may encode any desired product e.g. a protein, enzyme, polypeptide etc.

To express the desired genes, the expression vectors comprising the mutant Pm or Pu promoter systems of the invention conveniently contain one or more sites for insertion of a cloned gene, e.g. one or more restriction sites, located downstream of the promoter region. Preferably, multiple, e.g. at least 2 or 3, up to 20 or more, such insertion sites are contained. Vectors containing multiple restriction sites have been constructed, containing e.g. 20 unique sites in a polylinker. Suitable cloning sites for insertion of a desired gene are well known in the art and widely described in the literature, as are techniques for their construction and/or introduction into vectors (see e.g. Sambrook et al., supra).

For ease of construction, appropriate cloning sites may be introduced in the form of a polylinker sequence, using nucleic acid manipulation techniques which are standard in the art. A range of suitable polylinker sequences are known in the art and may simplify the routine use of the expression vectors containing a Pm/Pu mutant promoter. Thus, for example a well-known polylinker/lacZ' region may be used, as described for example in the vectors of Ditta et al., 1985, Plasmid, 13, 149-153, simplifying standard cloning procedures and identification of plasmids with inserts, by using the blue/white selection technique based on lacZ, which is well-known in selection procedures.

A number of other features may also be included in the mutant Pm or Pu containing vectors of the invention. Thus, the vectors may include features which assist in plasmid transfer, such as the oriT function if RK2 plasmid based vectors are used, which facilitates conjugation and is useful in cases where transformation/electroporation is inefficient, or if very high transfer frequencies are required.

As mentioned above, the reliable levels of expression and tightly controlled nature of such expression obtainable using the mutant Pm/Pu system across a broad host range, makes the expression vectors of the present invention particularly useful as tools for maximising and/or controlling expression of a desired gene product. A mutant Pm or Pu expression system may also be used for expression studies and physiological analyses in bacteria, for example to analyse metabolic pathways, e.g. determine rate limiting steps, conveniently also at intermediate or low expression levels, or for studies of plasmid transfer and dispersal in natural environments.

The present invention includes mutant Pm or Pu promoters which have the further advantage of acting in a gene-independent way, and thus these represent a useful and valuable starting point for optimisation of other parameters such as translation efficiency, which is highly gene-dependent. Such mutants could also possibly reduce the requirements for high copy-number vectors, for example RK2, which are known to cause problems in many gram-negative bacterial species. It also seems possible that further improvements in transcription may be obtained by introducing mutations in the xylS activator protein or its binding site in the 40 to -70 region of the promoter.

Such “gene-independent” mutants may be regarded as a “transcriptional” mutant of Pm or Pu. However, as discussed above, although these represent a particularly preferred and advantageous embodiment, the invention is not limited to such mutants and, although less preferred, encompasses also mutants which may have gene-specific effects. As described in more detail in the Examples below, an example of such a mutant is a mutant comprising a duplication of the −10 region in Pm. Such a mutant is less preferred since the modified properties of the promoter may be gene-dependent i.e. gene-specific. Although the effects of this mutant are not fully understood, it is believed that the modification results in changes in the region corresponding to the 5′-non-translated region of the corresponding mRNA and these may have an effect on translation. Thus, the −10 duplication mutant of Pm may be regarded as a “translational” mutant since the effects of the modification are believed to be exerted through translational effects. This could take place, for example, through effects on mRNA folding, and different folding structures may be created affecting translation. Although less preferred, such “translational” mutants are included within the scope of the present invention.

The promoter mutants giving rise to reduced background expression levels may be useful for studies in which physiological levels of gene expression is important. This would typically be needed in experiments designed to study rate-limiting steps in biochemical pathways. Mutants with reduced background expression have previously been described by Kessler et al., supra but these mutations are in the −35 region, near the XylS binding site. By combining such −35 region mutants with those of the present invention, background expression might be reduced to very low levels and such an expression control system comprising a mutant Pm promoter exhibiting reduced background expression combined with a mutant xylS structural gene and/or a mutant XylS binding site exhibiting reduced background expression levels. Thus, an expression system comprising a combination of any of the above features constitutes another aspect of the present invention.

The mutant promoters of the present invention may also advantageously be used in the control of biosynthetic pathways.

The biosynthetic pathway may be any pathway involving one or more enzymic reactions for the synthesis of any desired molecule. The gene placed under the control of the promoter of the invention may be any desired gene, for example encoding an enzyme in the pathway, or a regulatory protein. This may for example be a gene encoding an enzyme catalysing a rate-limiting step, or an enzyme synthesizing an important intermediate etc. Particularly suited for control using the promoters of the invention, are steps in a biosynthetic pathway where either tight control of induction (i.e. low backround expression) or a low level of expression generally, would be advantageous.

A suitable biosynthetic pathway might be, for example, any pathway where a wild-type Pm or Pu promoter is too active, and reduced expression would be desirable.

Levels of expression may vary from host to host, and hence appropriate expression-down mutants may be isolated in the host of interest using the procedures described herein.

One example of the application of the mutant promoter system is in the control of the biosynthesis of xanthan gum, a commercially important polysaccharide, which is a natural product of Xanthomonas campestris. Xanthan formation accompanies the normal growth phase of X. campestris, increasing the viscosity of the growth medium as the concentration of the product increases. This is a problem because the increase in viscosity inhibits further growth of the micro-organisms by limiting the transfer of oxygen to this obligately aerobic organism, so that they never reach the maximum potential cell density and this reduces the economic viability of the process. Placing one gene, early in the xanthan biosynthetic pathway and essential for the production of xanthan, under the control of a tightly controlled, conditional promoter such as an expression-down Pm mutant showing reduced leakage expression, would in effect switch off the entire biosynthetic pathway, preventing the synthesis of xanthan until a desired cell density has been reached by the producer cells. Thus, production efficiency can be increased and profitability improved.

A similar principle may be used to induce or “switch on” other biochemical pathways, once a desired cell density has been reached, or at any other desired time during cell culture or growth.

In a further aspect therefore, the invention provides the use of a mutant Pm or Pu promoter as hereinbefore defined in the control of a biosynthetic pathway, wherein at least one structural gene in said pathway is placed under the regulatory control of the mutant Pm or Pu promoter.

An example of a biosynthetic pathway is the xanthan biosynthetic pathway and the preferred structural gene controlled by a mutant Pm promoter in this pathway is XanA which encodes the bifunctional glucose and mannose phosphoglucomutase (Köplin et al., 1992, J. Bacteriol. 174, 191-199). If background activity (in the absence of inducer) of the controlling enzyme is too high, a mutant promoter with lower background activity is highly advantageous.

Clearly different mutant Pm or Pu promoter systems may have differing expression levels, degree of leakiness etc. in different species of host cells. Thus, to identify appropriate promoters for particular applications, it is desirable to be able to assess the properties of each promoter by screening a library of promoters in the host cell of interest.

In yet another aspect, the present invention provides a method for qualitatively, quantitatively or semi-quantitatively assaying promoter activity. In such a method an antibiotic resistance gene, for example the bla gene which encodes β-lactamase, is expressed under the control of the test promoter, which may be, for example, a mutant Pm promoter, and host cells are transformed with a vector comprising the antibiotic resistance gene thus controlled. The transformed cells can then be plated or inoculated onto or into media containing different concentrations of the antibiotic in question, in the case of bla, ampicillin or other penicillin derivatives. The level of promoter activity under any given condition determines the level of expression of the antibiotic resistance gene, e.g. of β-lactamase from the bla gene, and the level of expressed gene product e.g. β-lactamase determines the capacity for cell growth in the presence of such antibiotics, allowing promoter activity to be determined or assayed under different conditions etc. in the induced and/or uninduced states. Such a promoter screening activity is in essence suitable for the screening of any promoter, in any vector and in any suitable host cell susceptible to the antibiotic in question e.g. to β-lactam antibiotics. The method may conveniently be used to screen libraries of promoters under different conditions allowing the selection of promoters with the desired characteristics under the specific conditions e.g. of the characterisation of individual mutant Pm or Pu promoters in different host cells types, using different physical parameters e.g. temperature, inducers, different growth media etc. As an example, the plasmid pJT19bla was used herein to screen the mutant Pm promoter systems of the present invention. The only requirement for host cells in this aspect of the invention is that they are sensitive to the antibiotic used to determine the level of promoter controlled expression.

Thus, this aspect of the invention provides a method for assaying promoter activity, said method comprising expressing in an antibiotic-susceptible (i.e. sensitive) host cell, an antibiotic resistance gene under the control of the promoter to be assayed, and assessing the growth of said cell in the presence of said antibiotic.

Sources of antibiotic resistance genes or markers, and appropriate host cells are welt known in the art.

In a preferred embodiment, the antibiotic is a β-lactam antibiotic and the antibiotic resistance gene is a bla gene. Other antibiotic resistance markers which give a resistance increase in proportion to the amount of gene product expressed, e.g. chloramphenicol acetyl transferase, are described in Uhlin and Nordstroem, 1977, Plasmid, 1, 1-7.

The method of this aspect of the invention may be practised using techiques and materials well known and standard in the art. Thus, for example, an expression system or vector (e.g. a plasmid) may be created comprising the antibiotic resistance gene and a site for insertion of the test promoter in such a manner that it controls expression of the antibiotic resistance gene using DNA manipulation techniques well known in the art. An expression cassette comprising the test promoter (or a site for insertion thereof) and the resistance gene may be introduced into any convenient replicon. Any bacterial replicon may be used, although a low copy number system is advantageous, to reduce the effects of any backround transcription (in the absence of inducer).

Thus using the method of the invention, the activity of a mutant or modified promoter can be assessed, and, if desired, compared to wild-type. The activity can be assessed under induced and/or non-induced conditions, thus enabling the “leakage expression” under the promoter to be assessed.

The term “assaying” as used herein includes any assessment of the level or amount of expression under the promoter, whether relative or absolute. Thus, qualitative, quantitative and semi-quantitative assessments are covered.

The invention will now be described in more detail in the following Examples, with reference to the following drawings:

FIG. 1. Map of the plasmid pJT19bla.

The restriction enzyme sites shown are unique. Pneo, promoter for the neomycin phosphotransferase gene; bla, gene coding for β-lactamase; kan, kanamycin resistance gene; t, bidirectional transcriptional terminator; trfA, gene encoding the essential replication protein; oriV, origin of vegetative replication; oriT, origin of transfer. The transcriptional and translational part of the Pm promoter region is displayed above the plasmid map as described by Kaldalu et al., 1996, Mol. Microbiol. 20: 569-579 and Kessler et al., 1993, J. Mol. Biol. 230: 699-703. See Table 1 for details regarding the steps involved in the construction of pJT19.

FIG. 2. Map of mutants giving enhanced or reduced expression levels compared with wild type Pm. The transcriptional start site is indicated with an arrow. The eleven mutagenised bases are boxed and the introduced mutations are underlined. (The sequence of the pJT19U2002 is displayed on two lines to indicate the repeated parts).

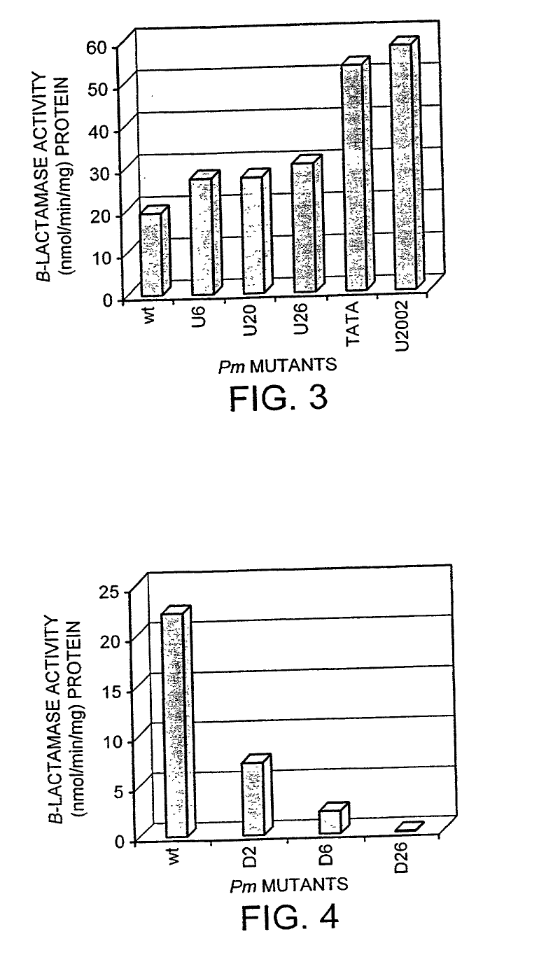

FIG. 3. β-lactamase expression from Pm mutants displaying enhanced expression levels. Over-night E. coli DH5α cell cultures containing the different Pm promoter mutant constructs were diluted 100-fold and grown exponentially to an OD₆₆₀ of 0.1. m-toluate was then added at 2 mM. β-lactamase activities were determined 5 hours after addition of the inducer.

FIG. 4. β-lactamase expression Pm mutants displaying reduced expression levels. The experiment was carried out as described in the legend to FIG. 3.

### EXAMPLES

### Example 1

### Generation of Pm Mutants Using Doped Oligonucleotide Mutagenesis

Materials and Methods

Bacterial strains, plasmids, and growth media. Bacterial strains and plasmids used in this study are described in Table 1. E. coli DH5α was used for plasmid propagation, cloning experiments, and for expression of β-lactamase and Luciferase, whereas the phosphoglucomutase-deficient E. coli strain W1485 pgmΔ: :tet was used for expression of celB. The E. coli strain JM109 was used for enrichment of single-stranded plasmid DNA and E. coli BMH 17-81 mutS was used for the mutagenesis procedure. For isolation of single-stranded plasmid DNA, the JMl09 cells were grown in TYP-medium (Promega). For all other purposes both E. coli and P. aeruginosa strains were grown in L broth (10 g/l tryptone, 5 g/l yeast extract and 5 g/l NaCl) or on L agar. E. coli was grown at 37° C., except for in the screening of Pm mutants and in the expression studies, where 30° C. were used. P. aeruginosa was grown at 30° C. Antibiotics were used when relevant at the following concentrations: kanamycin, 50 μg/ml; ampicillin, 100 μg/ml; tetracycline 15 μg/ml; streptomycin, 2 mg/ml; unless otherwise stated.

TABLE 1
Bacterial Strains and Plasmids

| Bacterial strains or | | Source or |
|---|---|---|
| plasmids | Properties^{a} | reference |
| Bacterial strains | | |
| E. coli | | |
| DH5α | endA1 hsdR17 supE44 thi-1 λ⁻ recA1 gyrA96 | Bethesda |
| | relA1 ΔlacU169 (φ80dlacZΔM15) | Research |
| | | Laboratories |
| S17.1 | RP4 2-Tc::Mu-Km::Tn7 pro res mod⁺ | (Simon et al., |
| | | 1983) |
| W1485 pgmΔ::tet | pgm negative derivative of W1485. Tc^{r} | (Lu et al., |
| | | 1994) |
| JM109 | recA⁻; F, lacI^{g} | Promega |
| BMH 17-81 mutS | recA⁺, Tc^{r}, mismatch repair negative | Promega |
| P. aeruginosa | | |
| PAO1161S | Spontaneous streptomycin resistant derivative | Haugan et al., |
| | of PA01161 | 1995 |
| Plasmids | | |
| pJB656 | RK2 based expression vector containing the Pm | Blatny et al., |
| | promoter and the gene encoding the regulatory | 1997b |
| | protein XylS. Km^{r}. 7.1 kb. | |
| pJB658 | Derivative of pJB656 but with a NdeI site at the | Blatny et al., |
| | translation start of the Pm promoter. Ap^{r}. 6.8 kb. | 1997b |
| pJT1 | Derivative of pJB656 in which the 1.5 kb | This study |
| | fragment between the XbaI and SfiI sites was | |
| | exchanged with the same region in pJB658 in | |
| | order to introduce the NdeI site at the | |
| | translational start codon. Km^{r}. 7.1 kb. | |
| pJT19 | Derivative of pJT1 in which SpeI site was | This study |
| | introduced downstream of the transcriptional | |
| | start site by cloning the two annealed synthetic | |
| | oligonucleotides 2A and 2B into the XbaI/NdeI | |
| | sites. Km^{r}. 7.1 kb. | |
| pPM2 | Derivative of pBR322 in which a NdeI site was | Paul |
| | introduced at the start codon of bla. Tc^{r}. 4.4 kb. | McNicholas |
| pJT19bla | The 0.9 kb I PCR fragment, using | |
| | primers 1A and 1B, containing the bla gene from | |
| | pPM2 was introduced into the same sites in | |
| | pJT19. Km^{r}. 9 kb. | |
| pJB655cop251Mluc | RK2 expression vector with the 1.7 kb luc gene | Blatny et al., |
| | cloned downstream of Pm and with the | 1997b |
| | cop251M mutation in the trfA gene. Ap^{r}. 8.5 kb. | |
| pJT19cop251Mbla | The 2 kb PvuII/BamHI fragment from | This study |
| | pJB655cop251Mluc was replaced with the 2 kb | |
| | PvuII/BamHI fragment of pJT19bla. Km^{r}. 8 kb. | |
| pJT19TATA | Derivative of pJT19 in which the 32 bp XbaI/ | This study |
| | SpeI fragment was replaced by the annealed | |
| | synthetic oligonucleotide 3A and 3B introducing a | |
| | consensus σ⁷⁰ in the −10 region of the Pm | |
| | promoter. Km^{r}. 7.1 kb. | |
| pJT19TATAbla | Derivative of pJT19TATA in which the 1 kb | This study |
| | SpeI/KpnI fragment containing the bla gene | |
| | form pJT19bla was introduced into the SpeI/ | |
| | KpnI sites of pJT19TATA. Km^{r}. kb. | |
| pGEM-luc | Vector containing the luc gene. Ap^{r}. 4.9 kb. | Promega |
| pALTER-1 | Vector for in vitro mutagenesis. Tc^{r}. 5.7 kb | Promega |
| pALTERluc | The StuI site was converted to NotI in pGEM- | This study |
| | luc (step 1) before the 1.8 kb HindIII/SacI DNA | |
| | fragment containing the luc gene was cloned | |
| | into the polylinker region of pALTER-1 (step 2). | |
| | Tc^{r}. 7.5 kb. | |
| pALTERlucNdeI | A NdeI site was introduced at the start codon of | This study |
| | the luc gene by site-specific mutagenesis using | |
| | primer 9. Ap^{r}, Tc^{r}. 7.5 kb. | |
| pJT19luc | The 1.7 kb luc gene was cloned into pJT19 as a | This study |
| | NdeI/SacI fragment. Km^{r}. 8.8 kb. | |
| pJT49bla | Derivative of pJT19bla, with the 32 bp XbaI/ | |
| | SpeI fragment removed from the Pm promoter. | |
| | Km^{r}. 8 kb. | |
| pJT49luc | Derivative of pJT49bla in which the bla gene | This study |
| | was exchanged with the 1.8 kb luc gene using | |
| | NdeI and BamHI. Km^{r}. 8.8 kb. | |
| pJB658celB | Derivative of pJB658 for expression of celB from | Blatny et al., |
| | Pm. Ap^{r}. 8.7 kb. | |
| pJT19celB | The 1.9 kb celB gene was cloned as a NdeI/ | This study |
| | BamHI fragment from pJB658 into pJT19. | |
| | Km^{r}. 9.0 kb. | |
| pML14 | A pACYC177 based plasmid vector containing | Lu et al., |
| | the pgm gene including its own promoter. | 1994 |
| | Ap^{r}. Km^{r}. 5.8 kb. | |
| pT7-7 | ColE1 based vector containing the T7 RNA | Tabor et al., |
| | polymerase promoter φ10 and rbs-site upstream | 1985 |
| | of the M13mp10 polylinker. Ap^{r}. 2.4 kb. | |
| pT7-7pgm | The 1.7 kb NdeI/EcoRI pgm gene from pML14 | This study |
| | cloned into pT7-7 by PCR (primer 10A and | |
| | 10B). Ap^{r}. 4.1 kb. | |
| pJB658pgm | The 1.7 NdeI/BamHI pgm gene cloned from | This study |
| | pT7-7pgm into pJB658. Ap^{r}. 8.5 kb. | |

^{a}Ap^{r}, ampicillin resistance; Km^{r}, kanamycin resistance; Tc^{r}, tetracycline resistance

DNA transformation, and conjugation. For cloning experiments plasmid DNA was introduced into E. coli by chemical transformation (Chung et al., One-step preparation of competent Escherichia coli: Transformation and storage of bacterial cells in the same solution. Proc. Natl. Acad. Sci USA, 86: 2172-2175, 1989). The Pm mutant bank was introduced into E. coli DH5α: by electrotransformation (Hanahan et al., Plasmid transformation of Escherichia coli and other bacteria. Meth Enzymol, 204: 63-113, 1991). Plasmids were transferred from E. coli S17.1 to P. aeruginosa by mating (Blatny et al., Improved broad-host-range RK2 vectors useful for high and low regulated gene expression levels in Gram-negative bacteria, Plasmid, 38: 35-51, 1997b). Selection of P. aeruginosa transconjugants were performed on agar medium containing kanamycin and streptomycin.

DNA manipulations. Plasmid DNA was prepared by the QIAGEN Midi-isolation Kit (Qiagen) for sequencing, the Wizard®Plus SV Minipreps DNA purification system (Promega) for cloning purposes and the Wizard Mini preparation Kit (Promega) for clonal analysis, as described by the manufacturer. DNA was extracted from agarose gel slabs using the QIAEX Kit (Qiagen). Other routine DNA manipulation was performed according to standard procedures (Sambrook et al., supra). The PCR reactions were performed using Taq DNA polymerase (Boehringer Mannheim) for cloning purposes and for screening of Pm promoter region sizes Taq DNA Polymerase from Promega was used. DNA sequencing reactions were performed by automated sequencing using the ABI PRISM™ Dye Terminator Cycle Sequencing Ready Reaction Kit (Perkin Elmer) Introduction of the NdeI site at the translational start site in the luc gene was done by site-specific mutagenesis, as described by the manual for “Altered Sites II in vitro Mutagenesis Systems” (Promega) Primers and oligonucleotides used in this study are described in Table 2.

TABLE 2
Primers and oligonucleotides

| No. | Primers and oligonucleotides | Applications |
|---|---|---|
| 1A | 5′GCAATTTAACTGTGAT3′ | Cloning of bla gene from pPM2 (pBR322 |
| | | HindIII ccw, Promega) |
| 1B | 5′TCCTGGTACCTTTTCTAC | Cloning of bla gene, introduction of KpnI |
| | GGGGTCTGA3′ | site downstream of bla in pPM2 (cw) |
| 2A | 5′CTAGAAAGGCCTACCCCTT | Introduction of SpeI site downstream of |
| | AGGCTTTATGCAACTAGTAC | Pm (sense strand) |
| | AATAATAATGGAGTCATGCA3′ | |
| 2B | 5′TATGCATGACTCCATTATTA | Introduction of SpeI downstream of Pm |
| | TTGTACTAGTTGCATAAAGCC | (antisense strand) |
| | TAAGGGGTAGGCCTTT3′ | |
| 3A | 5′CTAGTTGCATAAATTATAA | Introduction of σ⁷⁰ consensus E. coli |
| | GGGGTAGGCCTTT3′ | region into Pm (antisense strand) |
| 3B | 5′CTAGAAAGGCCTACCCCTT | Screening insertions of single Pm |
| | ATAATTTATGCAA3′ | promoters (cw) and Pm promoter |
| | | sequencing. Upstream of Pm |
| 4A | 5′AAGAAGCGGATACAGGAG | Screening of Pm promoters (cw). |
| | TG3′ | Sequencing primer for Pm mutants. |
| | | Located upstream of Pm. |
| 4B | 5′CTCAAGGATCTTACCGCT | Screening insertions of single pM |
| | GT3′ | promoters in bla (ccw) |
| 5 | 5′GGGTGAGCAAAAACAGGAA | Sequencing primer for Pm mutants in |
| | G3′ | bla (ccw) |
| 6 | 5′TTGTACTAGTTGCATATAG | Designed mutant pJT19U26 (ccw) |
| | CTTAAGGG3′ | |
| 7 | 5′TTGTACTAGTTGCATAAAA | Designed mutant pJT19D26 (ccw) |
| | CGTAAGGG3′ | |
| 8 | 5′TACTACGCACATTGGCACT | Used for introduction of designed |
| | G3′ | mutants (cw) |
| 9 | 5′CGTCTTCCATATGGATCC | Introduction of NdeI site at ATG of luc |
| | GGG3′ | by site directed mutagenesis |
| 10A | 5′AAAGGACAAACATATGGCAA | Used for cloning of the pgm gene from |
| | TCCACAATC3′ | pML14 into pT7-7 (cw). Introduction of |
| | | NdeI at ATG site of pgm |
| 10B | 5′ATCAGGGAATTCTGTGTTT | Cloning of pgm gene from pML14 into |
| | GTCATA3′ | pT7-7 (ccw). Introduction of EcoRI site |
| | | downstream of pgm |
| 11A | 5′CTAGTTGCATAAAGCCT | For doped oligonucleotide cassette |
| | AAGGGGTAGGCCTTT3′ | mutagenesis (antisense strand) |
| 11B^{a} | 5′CTAGAAAGGCCTACCCCT | The doped oligonucleotide (sense strand) |
| | 41332444143CAA3′ | |

Mutations that differs from the wild type is underlined and new introduced restriction enzyme sites are shown in italics.
^{a}The number in the oligonucleotide indicates the doping percentage as described by the A, C, G and T pools. 1 = 87% A, 4.6% G, 4.6% G, 3.8T (A pool); 2 = 87% C, 4.3% G, 5.2% A, 3.5%T (C pool); 3 = 87% G, 4.3% C, 5.2% A, 3.5% T (G pool); 4 = 87% T, 5.2% A, 3.9% G, 3.9% C (T pool).

To introduce the Pm mutant bank into the vector system a SpeI site was made downstream of the transcriptional start in the Pm promoter using oligonucleotide 2A and 2B. The two oligonucleotides were annealed by mixing them at concentration of 1.25 pmol/μl in H₂O, heated to 95° C. for two minutes, and slowly annealed by cooling to 70° C. over a period of ten minutes. The solution was left at 70° C. for ten minutes before slowly reducing the temperature to 20° C. over a period of ten minutes. The annealed oligonucleotides created XbaI and NdeI sticky ends.

To introduce an E. coli consensus σ⁷⁰ recognition site into the −10 region of the Pm promoter two oligonucleotides (3A and 3B, Table 2) were annealed at a concentration of 50 pmol/μl by the procedure described above except that the annealing temperature was 63° C. The annealed oligonucleotides created XbaI and SpeI sticky ends.

Construction of the Pm mutant bank For the doped oligonucleotide cassette mutagenesis the complementary synthetic oligonucleotides 11A and 11B were annealed. The numbers in oligonculeotide 11B indicate the doping percentages as described by the A, C, G and T pools. The contamination percentage of each base varies to take into account that the probabilities of being incorporated are not equal for all bases. Annealing was performed as described above at a concentration of 1.25 pmol/μl of each oligonucleotide and with an annealing temperature of 60° C. The annealed oligonucleotides created XbaI and SpeI sticky ends and were ligated into dephosphorylated pJT19bla previously digested with XbaI and SpeI creating the same sticky ends. After transformation into DH5α, plasmids were isolated and digested with SpeI to reduce the number of concatemer oligonucleotides in the bank. The linear form of the plasmid was then isolated from an agarose gel, eliminating plasmids with oligonucleotides inserted in the wrong orientation and also some of the concatemer forms. The religated pJT19bla plasmids were transformed into E. coli DH5α and used as a source for screening of Pm mutants. The construction of a site other than SpeI would have eliminated the concatemers, but this site was still chosen because it allowed more easy manipulations for other purposes. It also appeared unlikely that concatemer forms would be a problem, because of the phenotypes selected in the screening.

Enzyme assays. For the expression studies, E. coli cells containing the relevant plasmids was diluted 100-fold from an overnight culture grown in selective media. At an OD₆₆₀=0.1, the cells were induced by m-toluate at a concentration of 2 mM unless otherwise stated. Aliquots of cells were harvested 5 hours after induction and diluted or concentrated as appropriate. The method used in the β-lactamase assay, is a modified version of Ross and O'Callaghan., β-lactamase assay, Meth. Enzymol, 43: 69-85, 1975, and Chervaux et al, Secretion of active β-lactamase to the medium by the Escherichia coli haemolysin transporter pathway, Mol Gen Genet, 249: 237-245, 1995. The cell free enzyme extracts were made by sonication in an enzyme reaction buffer (80 mM K₂HPO₄, 20 mM NaH₂PO₄, pH 7.3) and β-lactamase activities were measured at room temp for 3 minutes. Total protein concentrations were measured using PROTEIN ASSAY (BIORAD). The properties of all mutants were confirmed by recloning, resequencing, and repeating the β-lactamase measurements. The BglII (in xylS) and ApaLI (in bla) sites were used for the subcloning.

Measurements of CelB activities were performed according to Fjærvik et al., Complementation of cellulose-negative mutants of Acetobacter xylinum by the cloned structural gene for phosphoglucomutase. FEMS Microbial Lett, 77: 325-330, 1991. Luc activities were measured as described by Blatny et al., 1997b supra, using the Luciferase Assay system from Promega performed in a TD-20/20 Luminometer (Turner Design) All enzyme expression analyses were done in from two to seven times and the average measurements are stated in the Results Section (Examples 2 to 6).

Results

### Example 2

### Construction of a Plasmid Vector Useful for Identification of Pm Mutants Displaying Altered Expression Levels

To screen for Pm mutants affecting the promoter activity, a vector (pJT19bla) was created in which bla was inserted downstream of Pm (FIG. 1). In pJT19bla, four bases immediately downstream of the transcriptional start site were changed to generate a unique SpeI site, and this modified Pm sequence is defined herein as wild type throughout. The SpeI site was created so that a synthetically doped linker could easily be introduced for random mutagenesis of the sequence containing the −10 region of the Pm promoter. The kanamycin resistance gene in pJT19bla allowed selection of the plasmid without the need to assume any particular expression level from Pm.

To analyse the bla expression properties of pJT19bla, transformed cells were plated at varying ampicillin concentrations in the presence and absence of an inducer, and also measured the corresponding β-lactamase activities (Table 3). Uninduced cells grew on agar medium in the presence of up to 100 μg/ml ampicillin, while the corresponding maximal resistance for induced cells was about 3000 μg/ml. Comparatively, the enzyme activity in the uninduced cells was below detection level, while it could easily be measured in the induced cells (13 nmoles/min/mg total soluble protein). To further verify the properties of pJT19bla as a tool for screening promoter activity, the trfA copy-up mutation cop251M was inserted, and expression analyses showed that both the resistance levels and the measured β-lactamase activities were substantially higher than from the wild type plasmid (Table 3).

TABLE 3
β-lactamase expression from the Pm promoter
in DH5α(pJT19bla) and effects of enhanced plasmid copy
number

| | Maximal ampicillin | | β-lactamase activity^{b} | |
|---|---|---|---|---|
| | resistance^{a} (μg/ml) | | (nmol/min/mg protein) | |
| Plasmid | induced | uninduced | induced | uninduced |
| pJT19bla | 3000 | 100 | 13 | — |
| pJT19cop251Mbla | 9000 | 500 | 90 | — |

^{a}The cells were incubated overnight with or without 2 mM m-toluate present in the growth media, diluted and platet on agar plates medium containing various concentrations of ampicillin
^{b}The expression levels of bla were determined after 5 hours in the presence of 2 mM m-toluate

### Example 3

### Screening for Pm Mutants with Enhanced or Reduced Expression Levels

In order to screen for mutants with enhanced β-lactamase expression levels compared to the wild type Pm promoter, an aliquot of the mutant bank was mixed with L broth containing m-toluate and incubated at 30° C. for 5 hours. The cells were plated at about 100,000 cells per plate on agar containing inducer and various ampicillin concentrations. The plates were incubated at 30° C. overnight and inspected for growth. Candidates gowing at high ampicillin concentrations were then individually retested as described below.

Screening for reduced background expression levels were performed by inoculating an aliquot of the mutant bank in L broth and left for 1 hour with shaking at 37° C. before plating on L agar containing kanamycin. Individual colonies where then inoculated into L-broth with or without inducer present in microtiter plates NUNC). The cells were incubated at 30° C. overnight, diluted with a 96 pin replicator in the same growth media and plated on L-agar containing various ampicillin concentrations with or without inducer. The plates were incubated at 30° C. overnight and monitored for growth.

### Example 4

### Construction of a Pm Mutant Gene Library, and Identification of Mutants Displaying Enhanced Expression Levels

To construct a Pm mutant gene library, both DNA strands in the region from the naturally occurring XbaI site and down to the introduced SpeI site were synthetically made (FIG. 1) One of the strands was left uniform (wild type) while in the other strand an eleven bp stretch covering the −10 region was randomly mutagenized by the use of a mixed nucleotide pool. The two strands were annealed and then ligated into pJT19bla that had been digested with XbaI and SpeI. The ligation mixture was finally transformed into E. coli DH5α, using the kanamycin resistance gene as a selection marker. All transformants, around 22,000, were mixed and used as a library for screening of Pm mutants.

For selection of mutants giving rise to enhanced expression levels, the gene library was plated on agar medium containing from 6 to 9 mg/ml ampicillin and 2 mM m-toluate. The results of these experiments showed that approximately 1% of the cells grew at 6 mg/ml ampicillin, while about 0.15% grew at 8 mg/ml, and no colonies were observed at 9 mg/ml. The Pm region in the plasmids from randomly picked colonies growing at 6 or 8 mg/ml ampicillin were sequenced, and the results showed, surprisingly, that all tested mutants contained two copies of the synthetic oligonucleotide oriented as direct repeats. Many of the mutants contained mutations in one or both of the oligonuclotide copies, but doublet wild type sequences were also found (see mutant pJT19U2002, FIG. 2), and the corresponding cells were found to grow at 8 mg/ml ampicillin. Thus, the enhanced resistance was a direct result of the duplication, which becomes possible because the sticky ends generated by XbaI and SpeI are compatible.

As will be shown below, the enhanced expression levels of the doublet mutants are probably not the result of an effect on transcription, and the screening strategy was therefore changed to search for mutants lacking a duplication of the synthetic oligonucleotide. For this purpose colonies were picked from plates containing 5 mg/ml ampicillin, and each plasmid was first prescreened by PCR-amplification of the Pm promoter. The PCR primers were designed such that they would generate a product of either 230 or 257 bp, depending on whether a single or a double copy of the synthetic oligonucleotide was present. These two fragments could be separated from each other by agarose gel electrophoresis, and from the screening of 200 colonies, 4 plasmids gave rise to a 230 bp fragment and with reproducible enhanced ampicillin resistance levels were sequenced. This resulted in the identification of two different mutants, each of which occurred twice among the sequenced candidates. The two mutations were both single base pair substitutions in the 11 bp doped region, and the corresponding plasmids were designated pJT19U20 and pJT19U6 (FIG. 2). In both mutants an A was introduced, and in pJT19U6 the original base was a G in the −10 region, while in pJT19U20 the original base was a T located just downstream of the −10 region. Measurements of the corresponding β-lactamase activities (FIG. 3) showed that the duplication in pJT19U2002bla enhanced the expression level 3-fold compared to the wild type, while pJT19U20bla and pJT19U6bla (single base-pair substitutions) increased the level between 30 and 40%. Therefore, it was clear that the duplication resulted in a much stronger stimulation of expression than that of the single base-pair substitutions. The background activities (uninduced cells) were not within the detection limit for any of the three plasmid constructs.

The synthetic oligonucleotide library was designed is such that the majority of the mutants would carry none, or only one mutation, while two mutations or more would occur at lower frequencies. From the screening analysis only single mutations were found and it seemed possible that maximal transcription from Pm requires more than one base to be changed. To test this the mutations in pJT19U20 and pJT19U6 were combined into a double mutant by the use of PCR (FIG. 2). The subsequent construct, pJT19U26bla, expressed β-lactamase at a 55% higher level than that of wild type Pm (FIG. 3). As for the other constructs, no background expression was detectable. Thus, the two mutations are at least to some extent additive.

The Am promoter −10 region does not contain an E. coli consensus σ⁷⁰ region. Plasmid pJT19TATA was constructed (FIG. 2) to comprise a mutant Pm promoter having an E. coli consensus σ⁷⁰ binding site. The β-lactamase expression level from this plasmid was nearly three times as high as that of wild type Pm in the presence of an inducer, reaching almost the level of pJT19U2002 (FIG. 3). The background expression levels were in this case slightly above the minimum detection level (data not shown).

Recombinant gene expression is well known to vary a lot in the same vector system, depending on the particular characteristics of each gene. To analyse the effects of the Pm promoter mutants on genes other than bla, the luc gene was inserted as a reporter in all the mutant constructs. Both single base-pair substitution mutants isolated from the library, the corresponding designed double mutant and the E. coli σ⁷⁰ consensus −10 mutant gave rise to a stimulation of the expression levels similar to those observed for β-lactamase (Table 4). Since Luc activity can be measured at very low levels, the uninduced expression could also be monitored. The results showed that background expression was even more stimulated, such that the induced/uninduced ratio went down. This effect was particularly strong for pJT19TATA.

Surprisingly, expression from pJT19U2002luc did not follow the gene-independent pattern observed for all the other mutants, as the expression level was found to be about 40% lower than from the wild type plasmid. To study this discrepancy further we made two new constructs in which the gene (celB) encoding phosphoglucomutase was used as a reporter instead of bla or luc. In the first of these, pJT19TATAcelB, the expression level was, very significantly stimulated compared to wild type, while expression from pJT19U2002celB was reduced, as for luc (Table 3). It therefore appears that the oligonucleotide duplication stimulatory effect is strongly gene-dependent, while this is not the case for the base changes in or near the −10 region.

TABLE 4
Luc and CelB activity from Pm mutants with enhanced expression
levels in E. coli

| | Luc^{a} and CelB^{b} activity | | |
|---|---|---|---|
| Plasmid | induced | uninduced | ratio |
| pJT19luc | 21 000 | 100 | 210 |
| pJT19U6luc | 29 000 | 260 | 112 |
| pJT19U20luc | 30 000 | 220 | 137 |
| pJT19U26luc | 34 000 | 680 | 50 |
| pJT19TATAluc | 41 000 | 2800 | 15 |
| pJT19U2002luc | 15 000 | 55 | 273 |
| pJT19celB | 35 000 | 370 | 95 |
| pJT192002celB | 23 000 | 170 | 135 |
| pJT19TATAcelB | 47 000 | 1200 | 40 |

^{a}The host strain used for the Luc measurements were DH5α. The experiment was carried out as described in legend to FIG. 3 and Luc activities were determined after 5 hours. Luc is given as arbitrary units
^{b}The celB expression levels were determined after 5 hours using 0.5 mM m-toluate as inducer. CelB is given as nmole/min/mg protein. The host strain used was W1485 pgmΔ::tet

### Example 5

### Mutations Leading to Reduced Background Expression Levels

About 1500 colonies from agar medium containing kanamycin were picked and tested with respect to their ampicillin resistance levels under uninduced and induced conditions. The phenotypes of about 10% of the colonies were after rescreening found to be different from that of the wild type, and these variabilities affected either the background resistance levels, the induced resistance levels, or both. Eighty-six mutants with a reproducibly altered phenotype were studied further, and eleven of these were found to display reduced background and reasonably good induced expression levels. Further characterisation of these strains showed that the altered phenotypes of nine of them were caused by the insertion of two or three oligonucleotides, in varying orientations relative to each other. Some of these oligonucleotides also contained various mutations. These plasmids were not studied further, but the remaining two mutants were, from the sequencing analysis, found to have single mutations located in the −10 region of the Pm promoter (FIG. 2). In pJT19D2 a G had been substituted with a C, and in pJT19D6 a C was exchanged with a T. As for the mutants described above the background β-lactamase activities could not be directly measured due to the low sensitivity of the assay. However, the induced expression could, and were as expected, found to be quite significantly reduced compared to that of the wild type (FIG. 4). A double mutant of the two single base substitutions was also constructed by PCR, and the corresponding mutant plasmid, pJT19D26, expressed even lower induced levels of β-lactamase than the two mutants. It therefore follows that combination of the single mutations acts in an additive manner, as for those that led to enhanced expression levels.

To be able to measure the background expression levels directly, the bla gene was replaced with luc, generating pJT19D2luc, pJT19D6luc and pJT19D26luc. Under induced conditions a reduced expression level was observed for all three mutants, relative to wild type, and the pattern of reduction was similar to that observed for β-lactamase (Table 5). It follows that the mutations act in a gene-independent manner, which is a great advantage for the general use of these mutants in the control of gene expression. The background expression levels were also significantly reduced relative to wild type, but unexpectedly, the levels were similar for all three mutants. To analyse this further we also constructed a mutant in which the region between the XbaI and SpeI sites was removed completely. The corresponding plasmid pJT49luc was found to express Luc at a level that was similar to that of the three mutants (uninduced) under both induced and uninduced conditions (results not shown). This result indicates that most of the remaining background activities in the mutants probably originate from transcription initiated from a site upstream of Pm.

As a further confirmation of these results the bla gene was also replaced by celB, generating pJT19D2celB and pJT19D6celB. The phosphoglucomutase activities expressed from these plasmids were lower than from the wild type plasmid, both under induced and uninduced conditions, as for β-lactamase and Luc (Table 5).

TABLE 5
Luc and CelB activity for Pm mutants with reduced expression
levels in E. coli

| | Luc^{a} and CelB^{b} activity | | |
|---|---|---|---|
| Plasmid | induced | uninduced | ratio |
| pJT19luc | 20 000 | 120 | 167 |
| pJT19D2luc | 5 110 | 42 | 122 |
| pJT19D6luc | 4 900 | 45 | 109 |
| pJT19D26luc | 800 | 42 | 19 |
| pJT19celB | 48 000 | 231 | 207 |
| pJT19D2celB | 28 000 | 144 | 195 |
| pJT19D6celB | 6 500 | 100 | 65 |

^{a}The host strain used for the Luc measurements was DH5α. The experiment was carried out as described in legend to FIG. 3. Luc activities were measured after 5 hours induction using 2 mM toluate as an inducer and is given as arbitrary units
^{b}The host cells used for CelB measurements were W1485 pgmΔ::tet. The level of CelB expression were determined after 5 hours using 2 mM inducer concentration (m-toluate). CelB activities are given as nmole/min/mg protein.

### Example 6

### Expression of luc from Pm Mutants in P. aeruginosa

Since both RK2 replicons and Pm can be used in a variety of hosts other than E. coli the activity of the mutants described here was studied in P. aeruginosa. The plasmids pJT19U20luc and pJT19U6luc both expressed enhanced levels of Luc relative to wild type, thus displaying phenotypes very similar to those in E. coli (Table 6). Plasmid pJT19TATAluc also showed enhanced expression levels in P. aeruginosa, but the stimulation was, in contrast to in E. coli, somewhat lower than for the single base-pair substitution mutants except for the enhanced background expression.

The expression down mutant pJT19D2luc was also analyzed in P. aeruginosa and was found to express Luc at a slightly lower level than the wild type under induced and uninduced conditions. The effect of the mutation is thus less significant compared to in E. coli, demonstrating that the host environment also may affect the phenotypes of Pm mutants. Induced expression from pJT19U2002luc was somewhat enhanced compared to the wild type plasmid, while the background expression level was reduced. These results differ from E. coli, again indicating host dependency.

TABLE 6
Luc activities expressed in P. aeruginosa from Pm expression mutants

| | Luc activity^{a} | | |
|---|---|---|---|
| Plasmid | induced | uninduced | ratio |
| pJT19luc | 23 800 | 430 | 55 |
| pJT19U20luc | 32 000 | 600 | 53 |
| pJT19U6luc | 31 000 | 560 | 55 |
| pJT19TATAluc | 28 000 | 1000 | 28 |
| pJT19U2002luc | 24 800 | 270 | 92 |
| pJT19D2luc | 21 600 | 360 | 60 |

^{a}The experiment was carried out as described in the legend to FIG. 3, except that the cells were induced for 12 hours using 2 mM m-toluate as inducer. Luc activity is given as arbitrary units

1. An isolated DNA molecule comprising a promoter sequence, said promoter sequence being a Pm or a Pu promoter of a TOL plasmid, said promoter sequence having sequence modifications in its −10 region.

2. A DNA construct comprising a modified Pm or Pu promoter as defined in claim 1 together with a corresponding regulatory gene xylS or xylR.

3. The DNA construct as defined in claim 2 which is an expression cassette.

4. The molecule or construct as defined in any one of claims 1 to 3 wherein said sequence modifications are present in the region spanning the nucleotide sequence from −1 to −25 nucleotides upstream of the transcriptional start site of said promoter sequence.

5. The molecule or construct as defined in any one of claims 1 to 4 wherein said sequence modifications comprise 1 to 6 base changes which may be contiguous or non-contiguous.

6. The molecule or construct as defined in claim 4 wherein said sequence modifications in the −10 region are as set out in FIG. 2, or as in any one of SEQ ID NO. 1 to SEQ ID NO. 8.

7. The molecule or construct as defined in any one of claims 1 to 6, wherein the mutant promoters have reduced or enhanced expression levels.

8. The molecule or construct as defined in any one of claims 1 to 7, wherein the mutant promoters result in reduced leakage or background levels of expression.

9. A process for preparing a mutant Pm or Pu promoter as defined in claim 1 comprising the step of addition, insertion, deletion or substitution of single or multiple nucleotides and/or inversion or repeat of two or more nucleotides in the −10 region thereof.

10. An expression vector comprising a Pm or Pu promoter mutant which exhibits a modified nucleotide sequence as defined in any one of claims 1 or claims 4 to 8.

11. The expression vector as defined in claim 8 wherein said vector is selected from the group consisting of a plasmid, virus, transposon, phagemid or phage-derived vector, or any other replicon.

12. The expression vector as defined in claim 10 or claim 11 wherein said vector exists or functions extrachromosomally in an autologously replicating form or is integrated into a chromosome.

13. The expression vector as defined in claim 10 wherein said vector is based on the RK2-based minimum replicon.

14. The expression vector as defined in any one of claims 10-13 wherein said vector comprises a transcriptional terminator inserted upstream of the modified Pm/Pu promoter.

15. A cell transformed with a vector as defined in any one of claims 10 to 14.

16. A method of expressing a desired gene within a host cell, comprising the steps of;
i) introducing into said cell an expression vector as defined in any one of claims 10 to 14, and containing said desired gene, and;
ii) culturing said cell under conditions in which said desired gene is expressed.

17. A method of preparing a desired polypeptide product encoded by a desired gene comprising the steps of;
i) culturing a host cell containing an expression vector as defined in any one of claims 10 to 14 into which the desired gene has been introduced under the control of the mutant Pm or Pu promoter, under conditions whereby said polypeptide is expressed, and;
ii) recovering said polypeptide thus produced.

18. Use of a mutant Pm or Pu promoter as defined in any one of claim 1 or claims 4 to 8, in the control of a biosynthetic pathway, wherein at least one structural gene in said pathway is placed under the regulatory control of the mutant Pm or Pu promoter.

19. A method for assaying promoter activity, said method comprising expressing in an antibiotic-susceptible (i.e. sensitive) host cell, an antibiotic resistance gene under the control of the promoter to be assayed, and assessing the growth of said cell in the presence of said antibiotic.
